(19) United States
(12) Patent Application Publication
(10) Pub. No.: US 2013/0274616 A1
(43) Pub. Date: Oct. 17, 2013

(54) ELECTROCARDIOGRAM DERIVED RESPIRATION SIGNAL FOR PARASYMPATHETIC AND SYMPATHETIC MONITORING DEVICES

(71) Applicant: THE ANSAR GROUP INC., (US)

(72) Inventors: Viprali V. BHATKAR, Cambridge, MA (US); Joseph Colombo, Richboro, PA (US); Robert Welch, Media, PA (US)

(73) Assignee: THE ANSAR GROUP INC., Philadelphia, PA (US)

(21) Appl. No.: 13/667,769

(22) Filed: Nov. 2, 2012

Related U.S. Application Data
(60) Provisional application No. 61/555,700, filed on Nov. 4, 2011.

(51) Int. Cl. A61B 5/00; A61B 5/0456; A61B 5/04; A61B 5/0205

(52) U.S. Cl. CPC A61B 5/4035; A61B 5/0205; A61B 5/0456; A61B 5/04017
USPC 600/484

(57) ABSTRACT

The invention presents a method for deriving respiratory data from single lead ECG recordings for monitoring the autonomic nervous system, specifically the parasympathetic and sympathetic nervous systems independently and simultaneously. The ECG derived respiration for ANS monitoring devices generally includes a method for non-invasive monitoring of the respiratory activity for the assessment of the parasympathetic and sympathetic (P&S) branches of the autonomic nervous system. The EDR signal is used to analyze and assess the individual activities of, and interactions between the sympathetic and parasympathetic divisions of the ANS. The present invention applies a QRS peak detection algorithm to ECG signal. The peak amplitudes and respective time locations are then used to generate the respiration signal. The EDR provides an approximate but reliable estimate of the respiratory activity. The utility of the algorithm is tested for the P&S monitoring for the various tasks such as Normal Breathing (Baseline), Deep Breathing, Valsalva Maneuvers, and Standing, as well as for normal subjects, and ill and geriatric patients.

### FIELD OF THE INVENTION

The present invention relates generally to Parasympathetic and Sympathetic (P&S) monitoring, also known as Autonomic Nervous System (ANS) monitoring and more specifically it relates to a method of electrocardiogram (ECG) derived respiration signal for P&S monitoring devices for deriving respiratory signal data from one or more lead ECG recordings for monitoring the autonomic nervous system, specifically the parasympathetic and sympathetic nervous systems independently and simultaneously.

### BACKGROUND OF THE INVENTION

The invention generally relates to the ANS monitoring which includes a method for non-invasive monitoring of the respiratory activity for the assessment of the parasympathetic and sympathetic (P&S) branches of the autonomic nervous system. The EDR (ECG Derived Respiration) signal is used to analyze and assess the individual activities of, and interactions between the sympathetic and parasympathetic divisions of the ANS. The present invention applies a QRS peak detection algorithm to ECG signal. The peak amplitudes and respective time locations are then used to generate the respiration signal. The EDR provides an approximate but reliable estimate of the respiratory activity. The utility of the algorithm is tested for P&S monitoring for the various tasks such as Normal Breathing (Baseline), Deep Breathing, Valsalva Maneuvers, and Standing for normal subjects and for ill and geriatric patients. There has thus been outlined, rather broadly, some of the features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

An object is to provide an ECG derived respiration for ANS monitoring devices for deriving respiratory data from single lead ECG recordings for monitoring the autonomic nervous system, specifically the parasympathetic and sympathetic nervous systems independently and simultaneously.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWING FIGURES

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a block diagram illustrating the overall process of an exemplary embodiment of a method for P&S monitoring process in accordance with the present disclosure.

FIG. 2 is a graphical illustration of an exemplary embodiment of a P&S monitoring system.

FIG. 3 is a waveform illustrating an ECG signal.

FIG. 4 illustrates a sample comparison of Sensor Acquired Respiration (SAR) and an EDR signal.

FIGS. 5A and 5B illustrate a sample comparison of P&S parameters for SAR and EDR.

FIG. 6 illustrates P&S parameters for SAR and EDR.

### DETAILED DESCRIPTION OF THE INVENTION

### A. Overview

Turning now descriptively to the drawings, in which similar reference characters denote similar elements throughout the several views, the figures illustrate a method for non-invasive monitoring of the respiratory activity for the assessment of the parasympathetic and sympathetic (P&S) branches of the autonomic nervous system. The EDR signal is used to analyze and assess the individual activities of, and interactions between the sympathetic and parasympathetic divisions of the ANS. The present invention applies a QRS peak detection algorithm to ECG signal. The peak amplitudes and respective time locations are then used to generate the respiration signal. The EDR provides an approximate but reliable estimate of the respiratory activity. The utility of the algorithm is tested for the P&S monitoring for the various tasks such as Normal Breathing (Baseline), Deep Breathing, Valsalva Maneuvers, and Standing, as well as for normal subjects, and ill and geriatric patients.

### B. ECG Derived Respiration

The present invention applies a QRS peak detection algorithm to ECG signal. The peak amplitudes and respective time locations are then used to generate the respiration signal. The EDR provides an approximate but reliable estimate of the respiratory activity. The utility of the algorithm is tested for the P&S monitoring for the various tasks such as Normal Breathing (Baseline), Deep Breathing, Valsalva Maneuvers, and Standing, as well as for normal subjects, and ill and geriatric patients.

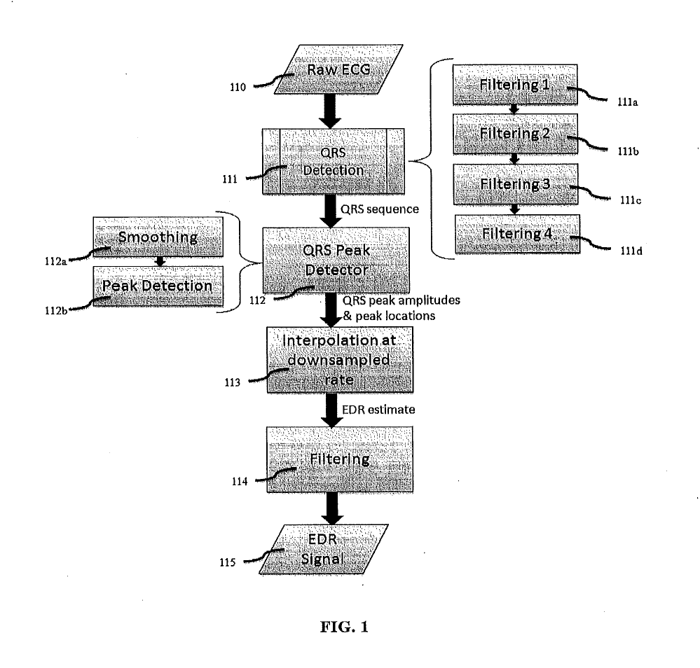

A flowchart shown in FIG. 1 illustrates the method of the present invention. The raw ECG is sampled at a minimum sampling rate of 250 samples per second. The first step of the method is to determine QRS signal (or R-peaks wave) (step 110). A R-wave detection algorithm from literature (described elsewhere) is implemented to obtain an approximate waveform of QRS peaks 111. In this step, ECG is first filtered using a band-pas filter 111a to reduce noise. The QRS signal is then generated by passing the filtered ECG through a series of operations, such as, differentiation 111b, squaring 111c, and a moving-window time averaging 111d, respectively. Next step is the QRS-peak detection 112. The QRS signal waveform produced in the previous step may contain ripples or multiple noise peaks. Hence, the QRS signal is first passed through a smoothing filter 112a before detecting peaks 112b. The QRS peak detector employs a simple local maxima peak detector of width 3. The R-peak amplitudes and their respective time locations are identified and are further processed to calculate 113 the EDR signal. A final cubic spline interpolation of these modulation amplitudes with respect to the peak time-locations yields continuous approximation for the EDR signal. A final low pass filter 114, for example a 5th order Butterworth filter with a frequency of 0.4 Hz, is used to reduce high frequency noise in the EDR signal 115. The final interpolation and filtering steps are performed at a down-sampling rate of 4 Hz as the respiration has very low frequencies (<0.5 Hz).

### C. Non-Invasive Real-Time P&S Monitoring Using HRV and ECG Derived Respiration

A method for non-invasive monitoring of the respiratory activity for the assessment of the parasympathetic and sympathetic (P&S) branches of the autonomic nervous system is presented. The EDR signal is used to analyze and assess the individual activities of, and interactions between, the sympathetic and parasympathetic divisions of the ANS.

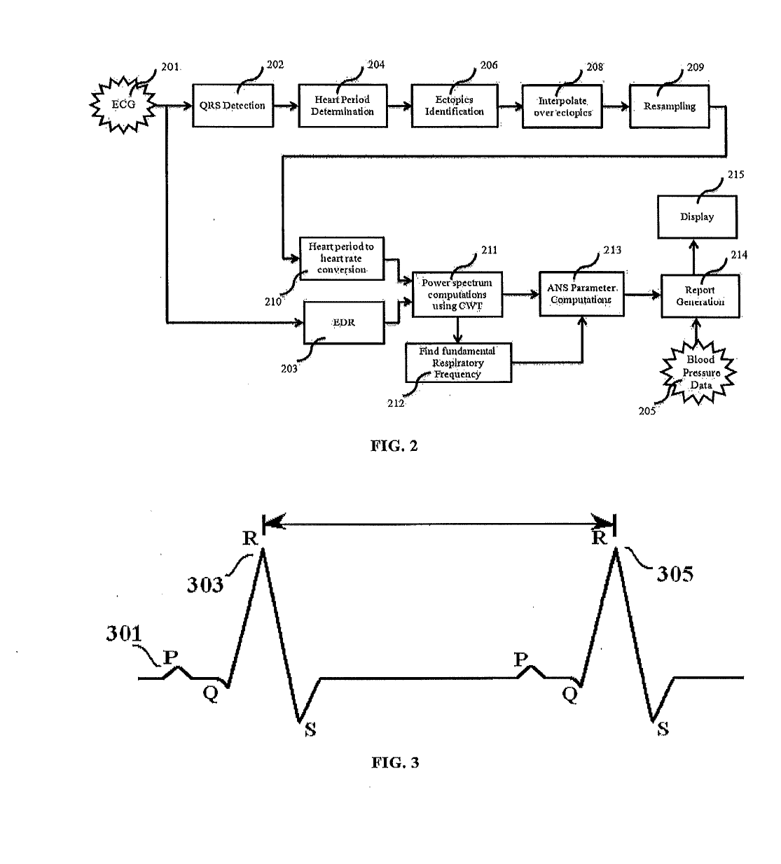

FIG. 2 is a graphical illustration of the system in accordance with an exemplary embodiment of the present P&S monitoring system. In the exemplary embodiment, input is gathered from three sources. The three input sources are the ECG source 201, the EDR source 203, and the blood pressure (BP) source 205. Methods to gather the data for ECG source 201 are well known in the art, and thus are not discussed herein in detail. The ECG source 201, which measures electrical impulses that stimulate the heart to contract, is sampled at a minimum sampling rate of 250 samples per second for example, and a more preferred rate of 1000 samples per second, so that the heart beat intervals can be measured precisely within a few milliseconds. The EDR source 203 provides respiration data extracted from ECG data and is discussed in detail elsewhere in this document. The BP source 205 monitors the patient's BP using a non-invasive BP measuring method such as the oscillometric method for burst assessment or a continuous method of assessment. The preferred embodiment for the continuous method is the Finapres method which provides the data required to perform a BP variability analysis using wavelet transforms in accordance with the present system. In the exemplary embodiment, each input signal is displayed in real-time on the output display 25 and is also processed concurrently.

The first step in conducting a hear rate analysis in accordance with the present system is to identify the fiducial point of the ECG signal, as well as the other defined points on the ECG signal (202). Here it is noted that reference numbers refer to both a step and a functional module of a computer, which might be a general purpose computer programmed to implement the specific algorithm represented by these steps to be a specific purpose computer having the corresponding modules. The fiducial point is the beginning point of movement of the heart that constitutes a heartbeat. An ECG signal can be represented by a waveform as shown in FIG. 3. The fiducial point on the wave corresponds to the start of atrial depolarization and is referred to as the P point 301. Atrial depolarization begins in the sinoatrial (SA) node which is controlled by the P&S nervous systems. The R peak 303 on the wave corresponds to the point of maximum ventricular depolarization. This nomenclature is well known in the art.

Next, the period of the heart is determined (204). The time between the onset of one heartbeat (P point) and the onset of the next heartbeat represents the period of the heart. However, because the R peak point is more easily identified than the P point, and the P-R interval is relatively constant in the absence of a conductive disorder of the heart, the generally accepted practice is to use the time interval between two consecutive R peaks (305) as the measure of the heart period. To identify the R peaks, the ECG signal is first filtered to reduce noise that could distort the wave. For example, the preferred embodiment uses a band-pass filter. The R peaks are then identified to produce a pulse train. For example, the preferred embodiment uses a differentiation and threshold algorithm. Applying the threshold algorithm to the differentiated pulse train identifies when the derivative exceeds a set threshold. Once the R peaks are identified, the time interval between the peaks can be computed by using the pulse train to start and reset a clock. The result is a sequence of R-R durations known in the art as the RR-interval tachogram.

The next step in processing the ECG signal in accordance with the present system is to identify any ectopics or missing beats (206). Electrical activity in the heart can affect heart rate variability analysis by causing abnormal wave formation. It is important not to confuse these disturbances with the modulation signal from the brain to the SA node. Thus, these erroneous signals needs to be removed before performing the spectral analysis on the P wave. It is possible to correct for these disturbances. For example, preferred embodiment uses interpolation to correct for these disturbances. The detail of this example is as follows.

Premature beats are characterized by a short beat-to-beat interval, followed by a longer than normal beat-to-beat interval. This will produce a sharp transient in the instantaneous heart rate wave. These beats can be identified using a mathematical algorithm. For example, the function r(n) defines the R-R interval of the heart beat number n. The time of the nth heart beat is defined by the following:

T(n)=Sum{r(i)} where the summation is performed from i=0 to l=n.

If the ratio r(n)/r(n−1) is larger than (1+x) where x is a predetermined threshold, then 10 r(n) and r(n−1) are considered incorrect and tagged for correction.

Additionally, a R-R interval histogram can be used to identify incorrect beats. The R-R intervals associated with an incorrect beat are generally significantly shorter or significantly longer than the normal R-R intervals, and correspondingly fall outside the major concentration of the histogram. A histogram can be computed for every 30 successive R-R intervals. The 25th and 75th percentiles of the histogram are identified. A small central region (e.g., the 10th beat to the 20th beat) within the 30 R-R intervals is then examined. If an R-R interval is larger than the 75th percentile (plus a predetermined threshold) or smaller than the 25th percentile (less a predetermined threshold), the interval is deemed incorrect. These two techniques are combined to accurately identify incorrect, missing, or premature beats. Once identified, these errors can be automatically corrected by applying a interpolation process (208) using the correct R-R intervals and their corresponding t(n) as inputs. For example, in the preferred embodiment, the interpolations process is a spline interpolation. The signal is re-sampled using the interpolation results (209). This assures that these disturbances do not corrupt the spectral analysis, and provides that any subsequent spectral analysis is performed on an evenly sampled, discrete time signal as opposed to the original unevenly sampled R-R interval tachogram. In some embodiments, it is desired to convert the measurement of R-R intervals (heart period) into an instantaneous heart rate, expressed in bpm (step 210). This is accomplished by using the following relationship: Heart rate=60/heart period.

Respiration signal is derived from ECG data in step/module 203 in accordance with the present system. Then, EDR and IHR signal power spectrums are calculated. For example, the exemplary embodiment uses a Continuous Wavelet Transform (CWT), 211. A frequency corresponding to a dominant peak in the respiration spectrum is considered as a Fundamental Respiratory Frequency (FRF), and is computed in step/module 212. Once the spectra for respiration and heart rate are derived, P&S parameters are calculated (213). The (typically) higher frequency area representative of only parasympathetic activity is chosen to be proportional with FRF from from 0.65*FRF to 1.35*FRF and is called as a Respiratory Frequency Area (RFa). The (typically) lower frequency area representative of only sympathetic activity is chosen to be the remainder of the frequency band from 0.04 Hz to 0.1 Hz that is not associated with the RFa and is denoted as LFa. The ratio of LFa to RFa is called Sympathovagal Balance (SB). The RFa is an independent measure of absolute parasympathetic activity while LFa is an independent measure of absolute sympathetic activity. SB is a measure of the relative levels of P&S activity.

### D. Comparison of EDR with SAR

The respiration signals derived from ECG (EDR) is compared with the sensor acquired respiration signal (SAR) for different tasks such as Normal Breathing (NB), Deep Breathing (DB), Valsalva Maneuvers, and Stand. Last three challenges are separated by a NB period.

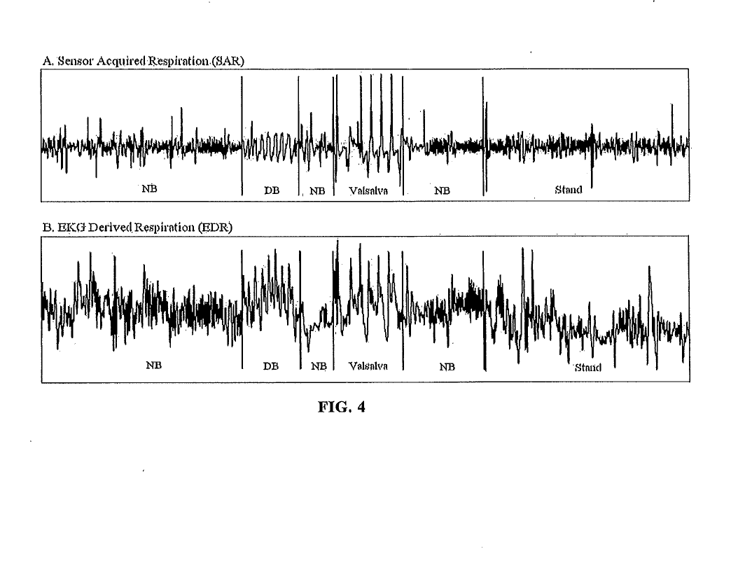

FIG. 4 depicts a sample visual comparison of the EDR signal (FIG. 4A) with the Sensor Acquired Respiration (SAR) signal (FIG. 4B). The data is obtained from a normal healthy mature adult. The SAR signal is acquired using the impedance pneumography. Both the SAR and EDR signals look very similar. As shown in the figure, the respiratory activity is monitored during a series of different tasks, Normal Breathing (NB), Deep Breathing (DB), Valsalva Maneuvers, and Stand. Last three challenges are separated by a NB period.

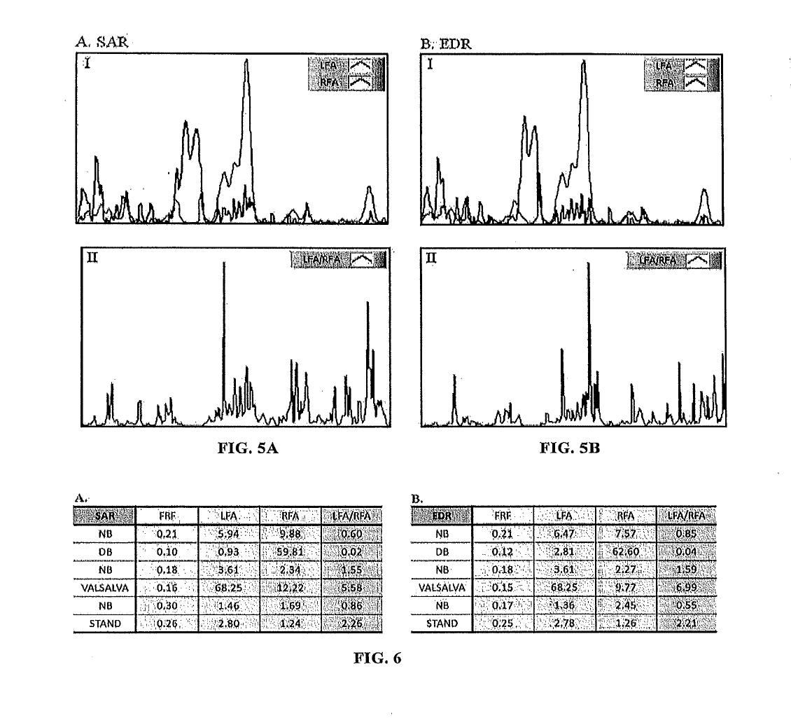

These signals also show similar P&S parameters and are presented in FIGS. 5A and 5B and Table 1. FIGS. 5A and 5B row I compares sympathetic (LFa) and parasympathetic (RFa) responses while row II compares SB (LFa/RFa) for SAR and EDR. FIGS. 5A and 5B show approximately same number of peaks in EDR based waveforms for P&S parameters agreeing with SAR based waveforms. The results of comparisons of the sets of parameters for SAR and EDR computed for different phases of the test are summarized in Tables shown in FIG. 6.

### E. Operation of Preferred Exemplary Embodiment

Traditional HVR-based measures are steeped in approximation and assumption. They are categorizes into two types, time-domain and frequency domain. Time-domain measures include statistical estimation of total autonomic activity such as mean, standard deviation (SD) of beat to beat heart rate (sdNN), root mean square of the SD (rmsSD), the percent of beat to beat greater than 50 ms (pNN50), a range of instantaneous heart rate (IHR) signal, and R-R interval plot. Statistical time domain measures are not appropriate for short data records as these values are affected by length of the data or the duration of the test. Also, these measures provide information on only one branch of ANS which is not very useful in assessing sympathovagal balance.

Other time domain measures include the exhalation-inhalation (E/I) ratio from a paced breathing event where the patient is breathing at 0.10 Hz, the Valsalva ratio, and the 30:15 ratio from a rapid, head-up, postural change event. These ratios are all measures of more or less HRV, which is only a qualitative measure of more or less parasympathetic activity. These measures are lacking any independent information regarding sympathetic activity.

Frequency domain measures are computed from spectral analysis of instantaneous heart rate (IHR) signal, or the related heart beat interval series, to identify oscillations in the signal associated with the autonomic activity. It is assumed that oscillations in the low frequency (LF) range, between 0.04-0.15 are associated with the sympathetic activity while the oscillations in the high frequency (HF range), 0.15 to 0.4 Hz are related to parasympathetic activity. Although, frequency domain measures provide information about both the branches of ANS, there is a problem with the traditional HRV analysis in isolating sympathetic and parasympathetic bands. Sometimes, LF band can correspond to both sympathetic and parasympathetic activities. For example, at lower respiratory rates, parasympathetic range shifts to LF range. Past research on ANS assessment has found that the changes in cardiac activity resulting from changes in sympathetic control cannot be interpreted accurately unless concurrent vagal activity is taken into account. Improper isolation of the ANS activities may end up in false-positives or false-negatives leading to misdiagnoses of autonomic disorders.

This demands a reliable technology for accurate assessment of the individual P&S branches of the ANS. A method of extracting respiration data from ECG signal provides a simple solution to the problem. This respiratory signal is called ECG-derived respiration or EDR signal. During normal resting conditions, the ECG of healthy individuals show periodic variation in R-R intervals. This variation is synchronized with respiration in which the R-R interval on an ECG is shortened during inspiration and prolonged during expiration. This rhythmic phenomenon is known as respiratory sinus arrhythmia (RSA). The modulation of amplitude of the ECG arises from the movement of the heart as we inhale and exhale. For example, with surface electrodes, the modulation of amplitude of surface ECG arises from the movement of electrodes with respect to the heart as we inhale and exhale. As a result, electrical impedance across the thoracic cavity changes with respect to each inhalation and exhalation modifying ECG amplitude. In another example, modulation of amplitude of intra-cardiac electrodes is also due to the motion of the heart with inhalation and exhalation. Our current technology successfully addressed the issue of isolating sympathetic and parasympathetic frequency intervals. It is not based only on spectral analysis of HRV but also used analyses of respiration to quantify the vagal activity. As respiration has a significant effect on vagal activity, the respiratory activity can be used in the frequency domain to correctly identify the frequency band corresponding to parasympathetic activity. A fundamental respiration frequency (FRF) is determined from the actual breathing rate and then the HF range is centered around FRF instead of a traditional fixed frequency range. This technology provides better and accurate measures of HRV by using respiratory activity to isolate frequency bands associated with the different P&S activities unlike traditional HRV method which cannot quantify these activities accurately with fixed bands. The present invention applies a QRS peak detection algorithm to ECG signal. The peak amplitudes and respective time locations are then used to generate the respiration signal. The EDR provides an approximate but reliable estimate of the respiratory activity. The utility of the algorithm is tested for the P&S monitoring for the various tasks such as Normal Breathing (Baseline), Deep Breathing, Valsalva Maneuvers, and Standing, as well as for normal subjects, and ill and geriatric patients.

P&S assessment technology as well as other applications like event monitors, holter monitors, loop recorder, and other ECG based technologies provide more information and clinically benefit from simultaneous tracking of ECG and respiration. Methods to obtain a respiratory signal include impedance sensors, pressure sensors, nasal thermocouples, etc. These traditionally used methods for ambulatory or home environment require transducer devices or sensors to be strapped to the chest or abdomen, or pressure transducers to measure nasal/mouth air pressure, each with associated burden of wear.

Although direct measurements are the most accurate, they may interfere with normal respiration. Some techniques may also require frequent calibration of the sensors.

Reducing number of sensors and processing required to perform these measurements is an important consideration. Throughout the medical community, including in the home-based environment, convenient technology solutions are needed that can avoid extra equipment and reduce processing time.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

1. A method for non-invasive monitoring of the respiratory activity for the assessment of the parasympathetic and sympathetic branches of the autonomic nervous system comprising the concurrent steps of:
a) deriving respiratory activity from the measured ECG signal; and
b) using ECG derived respiration (EDR) with heart rate to analyze sympathetic and parasympathetic divisions of the ANS.

2. A method set forth in step 1, wherein step a) further comprising the steps of:
a) detecting QRS complex from measured ECG;
b) determining R-peak amplitude;
c) interpolating peak amplitudes at the down-sampling rate of 4 Hz with respect to peak amplitude time locations; and
d) obtaining EDR estimate after low pass filtering the interpolated signal.

3. A method set forth in step 1, wherein step b) further comprising the steps of:
a) computing a first power spectrum from EDR signal;
b) determining the fundamental respiratory frequency from the first power spectrum;
c) obtaining instantaneous heart rate signals from ECG signals; and
d) computing a second power spectrum from instantaneous heart rate signals.

4. A method set forth in step 1, wherein step b) further comprising the steps of
a) locating said fundamental respiratory frequency from the first power spectrum in the second power spectrum;
b) identifying a respiratory frequency range around fundamental respiratory frequency in the second power spectrum;
c) computing a respiration frequency area from second power spectrum; and
d) computing a low frequency area from second power spectrum.

5. A method set forth in step 2, wherein step a) further comprising the steps of:
a) a first filtering of the raw ECG waveform to reduce influence of muscle noise, 60 Hz interference, baseline wander, and T-wave interference;
b) a second filtering to derive the QRS complex slope information;
c) a third filtering to identify the positive and amplified peaks; and
d) a fourth filtering to obtain an approximate QRS wave.

6. A method set forth in step 2, wherein step b) further comprising the steps of:
a) applying a filter to remove ripples and multiple noise peaks; and
b) computing R-peak amplitudes and their time locations.

7. A method set forth in step 4, wherein step c) further comprising the step of:
a) using respiratory frequency area (RFa) to determine a level of parasympathetic activity.

8. A method set forth in step 4, wherein step d) further comprising the step of:
a) using low frequency area (LFa) to determine a level of sympathetic activity.

9. A method set forth in step 1 wherein step b) further comprising the step of:
a) using the ratio of said level of sympathetic activity and said level of parasympathetic activity to determine a level of sympathovagal balance.

10. A method set forth in step 7, wherein step a) comprising the step of:
a) comparing said ratio with a set of existing standards.
